(19) United States
(12) Patent Application Publication
(10) Pub. No.: US 2020/0155872 A1
(43) Pub. Date: May 21, 2020

(54) LOW-INTENSITY PULSED ULTRASOUND FOR TREATMENT OF DEPRESSION

(71) Applicant: The Governors of the University of Alberta, Edmonton (CA)

(72) Inventors: Jie Chen, Edmonton (CA); Xinmin Li, Edmonton (CA)

(21) Appl. No.: 16/685,622

(22) Filed: Nov. 15, 2019

Related U.S. Application Data
(60) Provisional application No. 62/767,635, filed on Nov. 15, 2018.

(51) Int. Cl. A61N 7/00

(52) U.S. Cl. CPC A61N 7/00; A61N 2007/0026

(57) ABSTRACT

A portable therapy device for treating mental health disorders, syndromes or diseases includes (a) a wearable headband comprising at least one transducer element, comprising a piezoelectric crystal, a liquid coupling medium, and an elastomeric cap; and (b) a controller for causing the transducer to produce low-intensity pulsed ultrasound (LIPUS). A method of treating a mental health disorder, syndrome or disease in a mammal such as a human includes the step of applying LIPUS directly to the cranium of the mammal.

### FIELD OF THE INVENTION

The present invention relates to devices and methods for using low-intensity pulsed ultrasound in the treatment of depression and other mental health syndromes or diseases.

### BACKGROUND

Despite the extensive clinical application of antidepressants, 20%-40% of patients do not benefit sufficiently from the current antidepressants and 20% of patients are refractory to any antidepressant medication. Therefore, more effective treatment options must be developed. Ultrasound is an oscillating mechanical pressure wave with a frequency greater than the upper limit of the range of human hearing (˜20 kHz). In patients who do respond, there is often a 2-3 week or longer delay before clinical improvement becomes obvious (Taylor et al., 2006). Preliminary studies on effects of ultrasound on the central nervous system (CNS) have shown that low-intensity, low frequency ultrasound can noninvasively and remotely excite neurons and network activity by triggering voltage-gated sodium and calcium channels. Short-term application of transcranial pulsed ultrasound stimulation induces an increase in the density of brain-derived neurotrophic factor (BDNF)-positive puncta in hippocampus, indicating that ultrasound excites neuronal activity in mouse hippocampus and promotes endogenous brain plasticity. Transcranial focused ultrasound also induces an increase in the proliferation and differentiation of newborn cells and their survival as mature neurons. As the pathogenesis of depression may involve deficits of neurogenesis, loss of neurotrophic support and alteration of neuroplasticity, therapeutic ultrasound might be an alternative non-invasive antidepressant strategy.

As a result, there is a need in the art for alternative devices and methods of treating depression and related diseases or disorders.

### SUMMARY OF THE INVENTION

The present invention is based, at least in part, on the results of a study which investigated the application of low-intensity pulsed ultrasound (LIPUS), a specific type of ultrasound, as a therapeutic option for treating depression. LIPUS stimulation significantly increased the viability of both neuron-like SH-SY5Y cells and primary glia cells in vitro. Protein analysis revealed that LIPUS promoted the phosphorylation of β-catenin in primary glia cells and increased the level of brain-derived neurotrophic factor (BDNF) in both cell types. Subsequent animal studies using the repetitive restraint stress (RRS) model showed that LIPUS administration significantly alleviated the depression-like behaviors of mice in the sucrose preference test (SPT), tail suspension test (TST), forced swimming test (FST) and Y-maze test (YMT). Further testing indicated potential mechanisms for the beneficial effects of LIPUS on depression are associated with the promotion of neurogenesis and elevation of BDNF levels. With the cuprizone (CPZ)-induced demyelination animal model, it is demonstrated that LIPUS alleviated CPZ-induced damage to both mature myelin and oligodendrocyte progenitor cells (OPCs).

These findings indicate that LIPUS may be used as a therapeutic method to treat depression, not only by promoting neurogenesis and elevating BDNF levels, but also through protection and promotion of myelin and oligodendrocytes (OLs).

In another aspect, the invention may comprise a portable LIPUS therapy device. The portable feature allows patients to receive treatment anywhere and anytime. In some embodiments, the device may comprise:
(a) a wearable headband comprising at least one transducer element, comprising a piezoelectric crystal, a liquid coupling medium, and a cap, which is preferably elastomeric;
(b) a controller for producing pulsed ultrasound having an adjustable intensity of between about 30 mW/cm² to 150 mW/cm², at a frequency between about 1.0 MHz to about 2.0 MHz, with a pulse repetition rate of between about 0.5 kHz to about 2.0 kHz, and a duty cycle between about 10% and 50%.

### BRIEF DESCRIPTION OF THE DRAWINGS

In the drawings, like elements are assigned like reference numerals. The drawings are not necessarily to scale, with the emphasis instead placed upon the principles of the present invention. Additionally, each of the embodiments depicted are but one of a number of possible arrangements utilizing the fundamental concepts of the present invention.

FIG. 1A shows a Schematic Diagram of LIPUS Therapy Device. FIG. 1B shows a Schematic Diagram of LIPUS Therapy Device with the Hand held Unit.

FIG. 2 shows a schematic diagram of LIPUS Device System.

FIG. 3 shows an exemplary PCB Layout for Main Board (Top) and Drive Board (Lower Left Corner)

FIG. 4 shows one embodiment of a Transducer Cap from Three Different Angles (a) The View of Combined Case (b) The view of Male Case and Transducer (C) Inside View of the Female Case and Rubber

FIG. 5 shows an exemplary Setup for Measuring Acoustic Impedance.

FIG. 6 shows a graph of Sound Intensity Measurement and Attenuation Fitting Intensity of Sham Group and Rubber Group in Different Coupling Agents.

FIG. 7 shows a Comparison of Transmission Rate in different media.

FIG. 8A shows a LIPUS device and exemplary setup with a LIPUS generating box for a cell experiment, with a detailed view of the cell experiment; as well as a RRS with a mouse; LIPUS treatment of a mouse; and a detailed view of the LIPUS treatment of a mouse. FIG. 8B shows an experimental design for the in vitro cell study, for the in vivo animal study using the RRS model, and for the in vivo animal study using the CPZ model.

FIG. 9 shows graphs illustrating the effects of LIPUS on cell viability and the levels of pβ-catenin and BDNF in neuron-like SH-SY5Y cells and primary glia cells. a) LIPUS and the viability of SH-SY5Y cells. b) LIPUS and viability of primary glia cells. c) A representative Western blot image showing that LIPUS at the intensity of both 15 mW/cm² and 30 mW/cm² had no significant effect on the level of pβ-catenin in SH-SY5Y cells. The relative amounts of pβ-catenin protein in all groups are presented in the bar graph. d) A representative Western blot image showing the effects of LIPUS on levels of pβ-catenin in primary glia cells. The relative amounts of pβ-catenin protein in all groups are presented in the bar graph. e) A representative Western blot image showing effects of LIPUS on levels of BDNF in SH-SY5Y cells. f) A representative Western blot image showing effects LIPUS has on levels of BDNF in primary glia cells. The relative amounts of BDNF protein in all groups are presented in the bar graph. Values represent group mean values±S.E.M.. *p<0.05 vs. 0 mW/cm² (control), #p<0.05 vs. 15 mW/cm².

FIG. 10 shows graphs showing the effects of LIPUS on performance of mice in tests of depressive-like behaviors. a) Sucrose preference was decreased by RRS exposure and reversed by LIPUS treatment in the sucrose preference test. b) Total fluid consumption in the sucrose performance test was not significantly influenced by treatment in the sucrose preference test. c) There was a non-significant trend towards an increase in total time of immobility by RRS exposure and this immobility time was significantly decreased by LIPUS treatment in the tail suspension test. d) There was a non-significant trend towards an increase in total time of immobility by RRS exposure and this immobility time was significantly decreased by LIPUS treatment in the FST. e) There was a non-significant trend towards a decrease in spontaneous alterations by RRS exposure and this decrease was significantly reversed by LIPUS treatment in Y-maze test. f) No significant differences were observed in total number of arm entries as tested by the Y-maze test. Values represent group mean values±S.E.M.; n=8-9 mice per group. *p<0.05 vs. CTL; #p<0.05 vs. RRS.

FIG. 11 shows graphs showing LIPUS increased the levels of Doublecortin (DCX) and BDNF in mice exposed to RRS. a) A representative Western blot image showing the effects of LIPUS on the levels of DCX in mice with or without exposure to RRS. Relative amounts of DCX protein in all groups are presented in the bar graphs. b) A representative Western blot image showing the effects of LIPUS on the levels of BDNF. Relative amounts of BDNF protein in all groups are presented in the bar graphs. Values represent group mean values±S.E.M.; n=6 (for a) or 5 (for b) mice per group. *p<0.05 vs. CTL; #p<0.05 vs. RRS.

FIG. 12 shows the effects of LIPUS on levels of MBP and NG2 in mice exposed to CPZ. a) A representative Western blot image showing the effects of LIPUS on the levels of MBP was increased after exposure to CPZ. Relative amounts of MBP protein in both groups are presented in the bar graph. b) Representative immunohistochemistry staining images showing the effects of LIPUS on the levels of MBP. Relative amounts of MBP-positive labeling in corpus callosum in both groups are presented in the bar graph. c) A representative Western blot image showing the effects of LIPUS on the levels of NG2. Relative amounts of NG2 protein in both groups are presented in the bar graph below. d) Representative immunohistochemistry staining images showing the effects of LIPUS on the levels of NG2 after CPZ exposure. Relative amounts of NG2 positive labeling in corpus callosum in both groups are presented in the bar graph. Values represent group mean values±S.E.M.; n=3-4 mice per group. *p<0.05 vs. CPZ+Sham; **p<0.01 vs. CPZ+Sham. Bar, 150 μm (for b) or 100 μm (for d).

### DETAILED DESCRIPTION OF EMBODIMENTS

In one aspect, the invention comprises a wearable LIPUS treatment device. The device comprises a replaceable cap, preferably made of an elastomer, such as a polyurethane rubber, to cover a metal transducer. Polyurethane rubber is a preferred material for acoustic transmission if there is a suitable coupling agent inside the rubber cap. In some embodiments, the coupling agent may comprise purified water (such as Milli-Q™ water or Type I—ASTM D1193.91), transformer oil and/or ultrasound gel. Acoustic transmission coefficient studies show that purified water is a preferred coupling agent, with 39.76% transmission rate.

The device may communicate with other devices, such as by a computer network or the Internet. In some embodiments, a mobile device application installed on a mobile computer, such as a smart phone, may enable a user, such as a treating physician, to remotely control ultrasound dosage (intensity and duration). The mobile device may communicate with the treatment device by a wired or wireless connection, such as WiFi™ and/or Bluetooth™.

The ultrasound generator can drive the transducer by a suitable pulsed signal, such as a pulsed square signal generated from the impedance matching board. By embedding transducers and caps inside the headband, patients can simply place the LIPUS headband for the treatment via the left and the right temples. The ultrasound wave can transmit through the skull and excite the brain nerves in the hippocampus.

Commonly used ultrasound has a metal cap on the top of the piezoelectric crystal to prevent electrodes from being exposed directly to fluid or the body. Researchers have also investigated the influence of the flexural modes of piezoelectrically actuated metal caps on the beam widths and frequencies. However, as ultrasound is generated from the piezoelectric crystal, it is not suitable to use metal housing caps to touch the facial regions (especially for temple). In some embodiments, a replaceable elastomeric cap for the ultrasound transducer is suitable.

Four identical ultrasound transducers driven by the LIPUS device were used in the experiment to examine the transmission sound intensity with the cap design and different ultrasound coupling agents (sham group, purified water, transformer oil and ultrasound gel). A preferred material for the ultrasound output system was identified through data analysis and curve fitting.

By comparing the ultrasound intensity transmission rate, purified water is proved to be the best coupling agent material filling between the elastomeric cap (polyurethane rubber) and transducer/piezoelectric crystal.

A. Transducer Device Design and Circuit Optimization

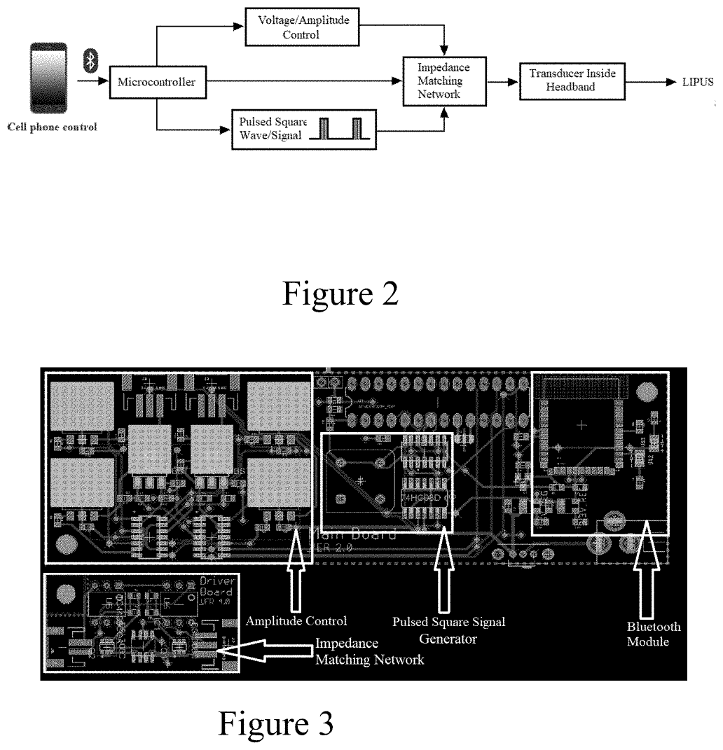

The preferred signal specifications of two-channel LIPUS generator device are: adjustable intensity of 30 mW/cm² to 150 mW/cm², 1.5 MHz frequency with pulse repetition rate of 1 kHz and 20% duty cycle. An exemplary module diagram is shown in FIG. 2 which may achieve these design specifications.

In FIG. 2, a microcontroller sets the amplitude and duration time of a pulsed square wave via the instruction sent by a mobile application on a mobile device. The connection between the mobile device and the microcontroller may be any wired or wireless protocol for sending and receiving data, such as Bluetooth communication. A piezoelectric transducer placed in the headband driven by pulsed square wave generates a LIPUS signal. Pulsed square wave impedance matching network allows sufficient delivery power to drive the piezoelectric transducer. Consequently, LIPUS passes through the rubber cap to treat desired function area of interest in brain.

FIG. 3 shows two PCB boards layout design comprising a main board and a driver board. The main board contains the Bluetooth module, a microcontroller, pulsed square signal generator and supplied voltage amplitude control. The driver board, comprising a impedance matching circuit, is placed inside the headphone next to the transducer to drive the transducer.

A wearable and remotely controllable LIPUS device may help patients receive LIPUS treatment conveniently. By using the app on the mobile phone, patients and doctors can remotely control and customize the therapy. The elastomeric transducer cap inside headband is suitable for mental disease therapy by decreasing both physical and psychological damage.

B. Ultrasound Cap Design

Acoustic transmission material can be either solid or fluid. The ideal sound transmission material allows sound waves to pass through to the sound-transmitting layer without any reflections. Therefore, the characteristic acoustic impedance of the material is preferably matched to that of the water. The attenuation constant of the material should be as small as possible. In this circumstance, polyurethane rubber is a preferred material due to its suitable acoustic characteristics.

Acoustic impedance Z of polyurethane rubber, the value of ρc (that is, the product of the density of rubber and the propagation velocity of sound waves in rubber) is matched with the ρc value of the sound wave propagation medium, e.g. water. Acoustic impedance is a property of material measuring how much acoustic pressure applied to the medium at corresponding acoustic vibration, which is usually presented as a product of the density p of the medium and the speed of sound C in the medium in g/cm2·s.

The acoustic energy loss is smaller when the sound waves pass through the rubber. It is known that rubber has a relatively lower insertion loss and high echo reduction in the various frequency range. Plastic deformation caused by acoustic waves leads to the sound energy attenuates. As the temperature increases (from 3.9° C. to 33.6° C.), both sound speed and sound attenuation of the frequency dependence decreases.

Acoustic incidents through the polyurethane rubber, the sound attenuation value depends on the rubber composition, which includes two parts: one is the choice of rubber species; the other is the choice of compounding agent.

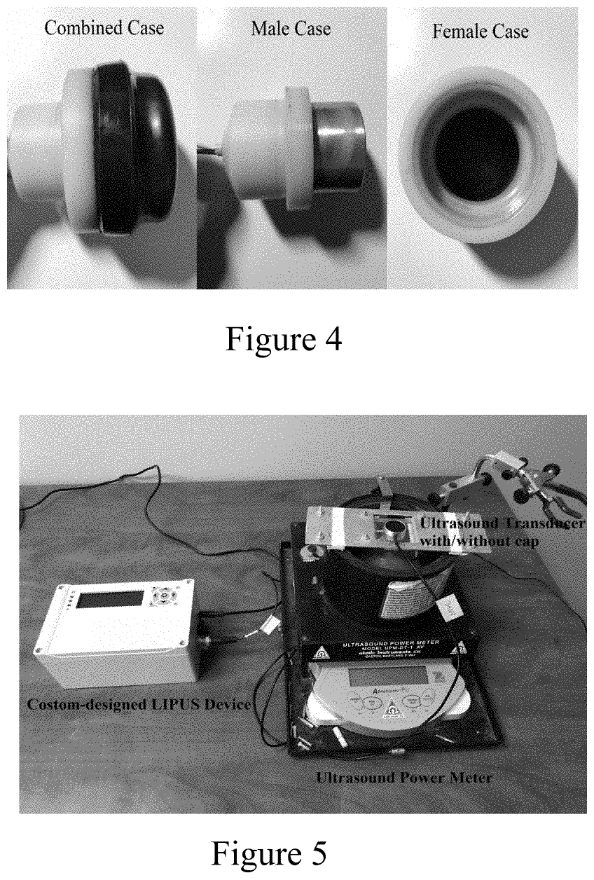

As shown in FIG. 4, in some embodiments, a plastic base is fixed at the bottom of transducer. The other side of the plastic is glued with polyurethane rubber which pouring as a cap with the thickness of about 1.6 mm. A detachable plastic cap is connected with rubber and fixed at the front of the transducer. The transmission cables of the transducer are designed at the back of the piezoelectric crystal, which provides space to build a separate case for the transducer. A spiral case is produced with the rubber jacketed on.

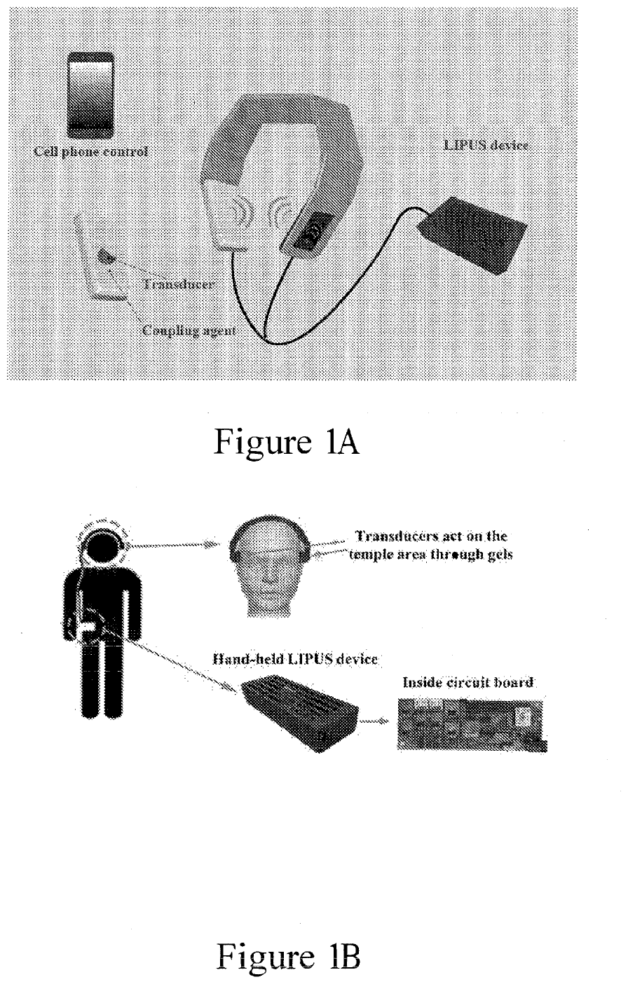

In some embodiments, as can be seen in FIG. 4B, the male case is stuck to the piezoelectric transducer and the female case is attached with a cylindrical rubber. The female case shown in FIG. 4C is filled with an ultrasound coupling agent and combined with the male case by threading them together, as shown in FIG. 4A. This design permits the female case to be disposed of with each use, to prevent cross-infection. This whole ultrasound transducer cap will be concealed within a head band to treat specific area (e.g. temple) as shown in FIG. 1.

In another aspect, the present invention comprises an application of low-intensity pulsed ultrasound (LIPUS) as a therapeutic option for treating depression and depression related effects. Without restriction to a theory, it is believed that LIPUS could have antidepressant effect in mammals including humans, with neurogenesis and neurotrophy as underlying mechanisms and that the protection and promotion of myelin and oligodendrocytes (OLs) might also be therapeutic targets for LIPUS. Although the monoamine hypothesis, which states that there is a functional deficiency of noradrenaline and/or serotonin (5-hydroxytryptamine, 5-HT) in certain parts of the brain in depression has dominated thinking about the etiology of depression for many years, it is obvious that other factors are also involved. Research in recent years has resulted in the development of hypotheses of depression focusing on disruption of neurogenesis in the hippocampus.

An in vitro cell study was first conducted to investigate whether LIPUS stimulation could increase neural cell viability, acting through neurogenesis-related cell signaling pathways. The Wnt-signaling pathway is important in neurogenesis, mediating neuroblast proliferation and neuronal differentiation in adult hippocampal progenitor cells via β-catenin, an intracellular signal transducer. Disruptions to this pathway have been reported in autism, schizophrenia and depression. DCX is a microtubule-associated protein expressed by neuronal precursor cells and immature neurons and is considered a marker for neurogenesis. BDNF is a neurotrophic factor whose expression has been reported to be increased in the rodent brain by several types of antidepressant therapy.

Two types of cultured cells, neuron-like SH-SY5Y cells and primary glia cells, were used and measurements of cell viability, β-catenin, DCX and BDNF were made. The interesting results obtained with these cells indicated that subsequent studies with animal models were warranted. We then applied LIPUS in an in vivo animal study using two models of depression, a repetitive restraint stress (RRS) model and a cuprizone (CPZ)-induced demyelination model. Behavioral tests [sucrose preference test (SPT), tail suspension test (TST), forced swimming test (FST) and Y-maze test (YMT)] were conducted to evaluate whether LIPUS could ameliorate animals depression-like behaviors. Follow-up protein analysis with Western blotting and immunohistochemistry (IHC) staining with regard to MBP and NG2 was performed to reveal whether the beneficial effects of LIPUS were associated with the promotion of neurogenesis and/or prevention of white matter deficits. MBP is a protein marker for mature OLs and is essential for the process of myelination. NG2 is a protein marker for OPCs that are capable of giving rise to new OLs under both normal and demyelinating conditions. To our knowledge, this is the first study focusing on the long-term effects of ultrasound in the CNS as a non-invasive therapeutic option for depression and employing both in vitro cell studies and in vivo animal studies.

Utility of the present invention is based, at least in part, on the demonstration that LIPUS increases the viability of neuron-like SH-SY5Y cells and primary glia cells in vitro and increases their levels of BDNF, as well as reduces depression-like symptoms and increasing brain levels of DCXand BDNF in mice exposed to RRS in mice exposed to RRS and attenuates neurobiological changes in mice exposed to both RRS and CPZ.

Transcranial ultrasound may be effective for neuromodulation. Ultrasound can directly modulate neuronal activity in the hippocampus, elicit action potentials in neurons, and stimulate the motor cortex in mice and the somatosensory cortex in humans. LIPUS, a specific type of ultrasound, has an arousing effect on several types of progenitor cells, including fresh hematopoietic stem/progenitor (HSP) cells, Chinese Hamster Ovary (CHO) cells, and osteoblast cells. Combined with the prompt effects of ultrasound on neurons and neural network activity, these studies imply that LIPUS might provide a stimulatory effect on CNS activity and be a therapeutic antidepressant option. A recent pilot study reported a potential association between ultrasound administration and elevated mood in humans⁹, and a study in rats using focused ultrasound in conjunction with circulating microbubbles to open the blood-brain barrier in the hippocampal region reported that after receiving two weekly treatments rats showed antidepressant-like effects (Mooney et al., 2018). The results of behavioral testing in the present study with the RRS model support this contention. In our study, we first conducted in vitro experiments using different cell types as a quick and efficient way to determine if ultrasound was exerting any beneficial effects that would justify further exploration using in vivo models. Histological testing revealed changes in markers of neurogenesis and BDNF in the presence of LIPUS treatment.

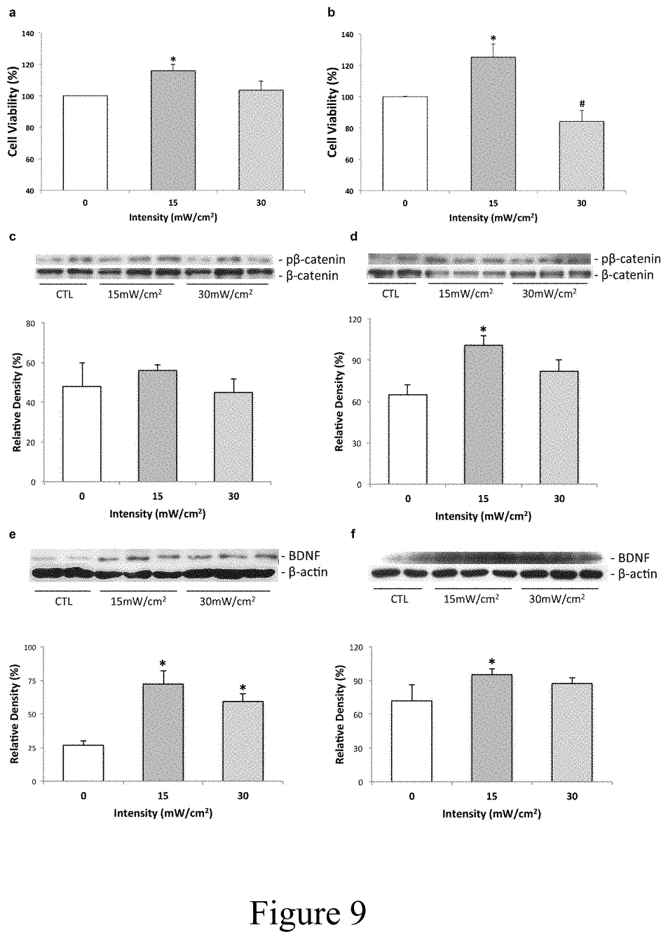

In some embodiments, LIPUS is ultrasound having an intensity lower than about 40 mW/cm². LIPUS at 15 and 30 mW/cm² was selected to test effects on cell viability. LIPUS at 15 mW/cm² accelerated SH-SY5Y cell growth (FIG. 9a). At 30 mW/cm², there was no change from control values. It appears that the growth of SH-SY5Y cells is only affected within a certain range of intensities, with around 15 mW/cm² having a significant impact. LIPUS increased the viability of primary glia cells (FIG. 9b). With the administration of 15 mW/cm², a significant increase in cell viability occurred. At 30 mW/cm², the number of cells was not significantly different from controls and lower than at 15 mW/cm². It is important to note that the effects of LIPUS stimulation on cell viability, phosphorylation of β-catenin and levels of BDNF are highly dependent on the cell type in terms of the intensity and duration that is required.

LIPUS at 15 mW/cm² elevated the phosphorylation of β-catenin in primary glia cells (FIG. 9d) and but had no effect on phosphorylation of β-catenin in neuron-like SH-SY5Y cells (FIG. 9c). This finding suggested that the positive effects of LIPUS on cell viability might be associated with activation of the Wnt pathway in these neural cells.

In our in vitro cell tests, LIPUS at 15 mW/cm² significantly increased levels of BDNF in both neuron-like SH-SY5Y cells (FIG. 9e) and primary glia cells (FIG. 9f), while LIPUS at 30 mW/cm² also induced a significant increase in levels of BDNF in SH-SY5Y cells but not in primary glia cells, indicating a different pattern of response to LIPUS stimulation between these two cell types.

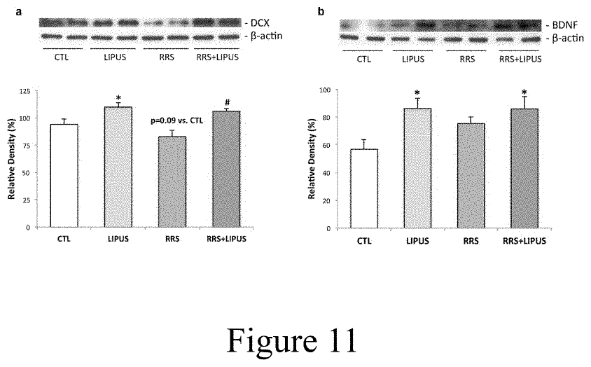

Interestingly, we did not observe a significant reduction of BDNF levels in mice exposed to RRS (FIG. 11b). Previous studies have shown that chronic restraint stress for 6 hours a day for 3 weeks did not change levels of hippocampal BDNF^{32, 33}. However, another study found that 1-hour daily restraint for 3 weeks led to a relative increase of plasma BDNF in animals³⁴. Further studies examining effects of LIPUS on BDNF expression using other animal models of depression induced by chronic stress, such as unpredictable chronic mild stress (UCMS), which is more correlated with actual human patients' experience than the predicted stress model³⁵, should be conducted in future to better understand the relationship.

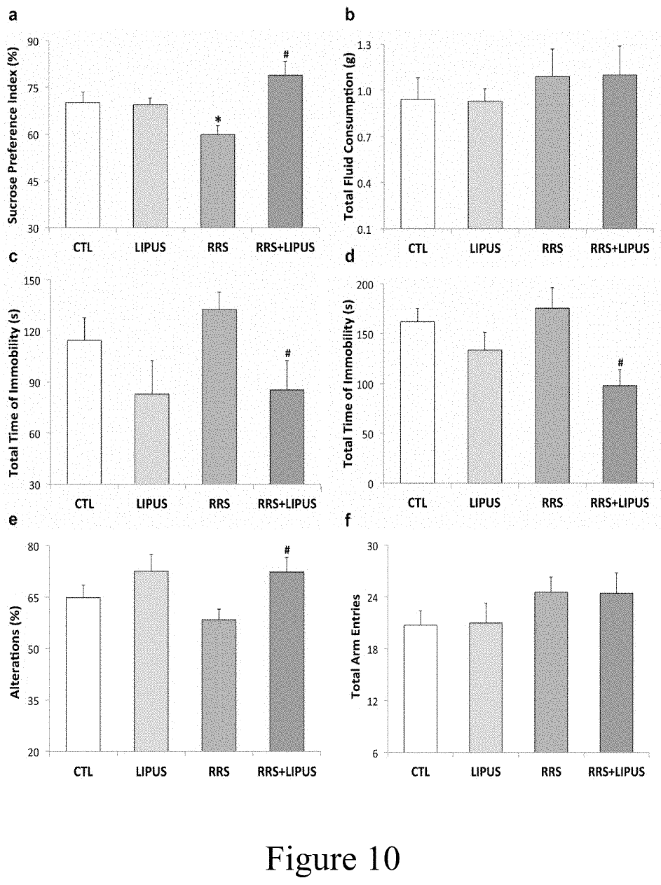

A key symptom of depression is anhedonia. Decreased intake of sucrose solution in the SPT is regarded as a behavioral measure of anhedonia in rodents³⁶. In the present study, mice exposed to RRS had a decreased sucrose preference index, while LIPUS treatment significantly reversed this decrease (FIG. 10a). Total fluid consumption across groups showed no differences (FIG. 10b). Consistent with our findings, previous studies have shown that chronic restraint stress induced a decrease in sucrose preference in animals³⁷, which could be reversed by the antidepressants fluoxetine and reboxetine³⁸. LIPUS also had an effect on lack of escape-related behavior associated with depression in the TST and the FST by decreasing the total time animals stayed immobile (FIGS. 3c and 3d). Both TST and FST³⁹ are used for screening for potential antidepressants, and previous studies have shown that antidepressants such as fluoxetine⁴⁰, venlafaxine⁴¹, bupropion⁴² and quetiapine⁴³, and neurostimulation therapies, including electroconvulsive therapy (ECT)⁴⁴, transcranial magnetic spectroscopy (TMS)⁴⁵ and deep brain stimulation (DBS)⁴⁶, reduce total time of immobility in the TST or FST. Spatial working memory was evaluated by the YMT. Mice exposed to RRS had a non-significant trend towards lower spontaneous alterations (FIG. 10e), and LIPUS treatment significantly improved performance in this test.

To further explore possible mechanisms underlying LIPUS effects on behavior, we investigated important mechanisms involved in the pathogenesis of depression. Substantial studies implicate deficits in neurogenesis in the development of depression^{47,48}. Most major types of antidepressant medications⁴⁹, as well as neurostimulation therapies like ECT and rTMS^{50,51} induce hippocampal neurogenesis in animals.

Mice exposed to RRS had a trend towards a significant decrease in levels of DCX (p=0.090) compared with the control group, while LIPUS treatment significantly reversed this decrease, a finding confirmed by the protein analysis of DCX (FIG. 11a). The LIPUS-only group had a significantly higher level of DCX than normal controls, indicating that LIPUS also promoted neurogenesis in regular animals (FIG. 11a). Similar to our findings, other research has shown that antidepressants increase hippocampal neurogenesis by promoting proliferation of adult hippocampal neural stem cells^{52,53}. Administration of ECT⁵⁰ and rTMS⁵¹ increases the number and dendritic complexity of adult-born migrating neuroblasts.

The CPZ model was studied to evaluate effects of LIPUS on myelin and OLs in depression. Previous studies have proposed that demyelination and impaired OL function may play a role in the etiology of depression^{54,55}, and depression is a common symptom in patients suffering from multiple sclerosis, a disorder involving demyelination⁵⁶. Our in vivo study showed that LIPUS did not significantly attenuate the depression-like behaviors of mice after exposure to CPZ, as tested by the TST, the FST, and the YMT. LIPUS could be considered a mild treatment, while CPZ is capable of producing extensive demyelination in the CNS and inducing severe behavioral and neurobiological dysfunction that LIPUS may not be able to ameliorate appreciably⁵⁷. Future studies should compare the anti-depressant efficacy of LIPUS with other standardized therapeutic options in this model.

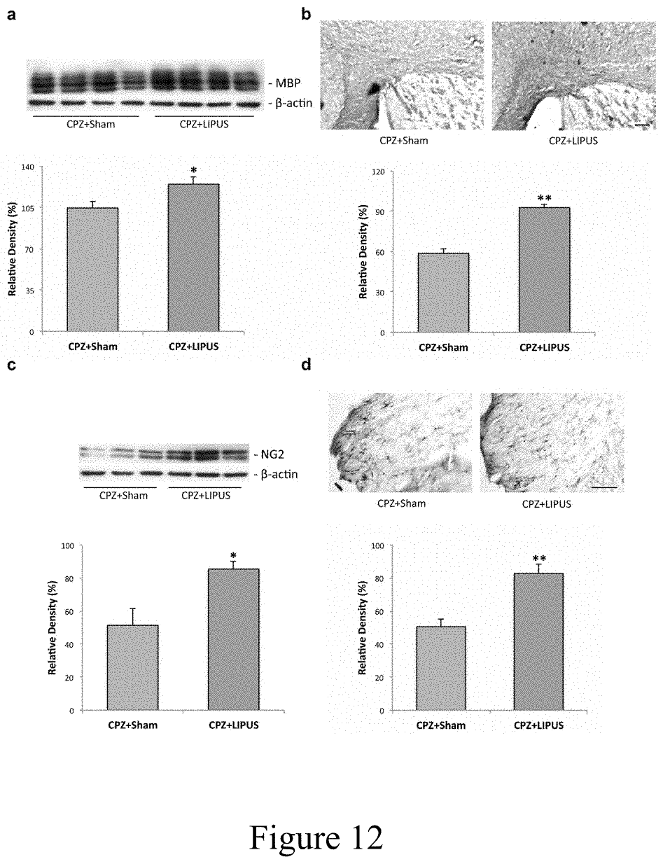

MBP plays a crucial role in the process of myelination and is a marker for mature OLs. We found that LIPUS significantly increased levels of MBP after CPZ exposure (FIGS. 12a and 5b), indicating a protective effect of LIPUS on mature myelin and OLs against CPZ toxicity. Our previous research found that MBP was significantly decreased in animals exposed to CPZ for 6 weeks⁵⁸; this decrease was reversed by chronic treatment with quetiapine, an antipsychotic also possessing antidepressant properties^{59,60}. Another study showed the CPZ-induced decrease in MBP was attenuated by chronic treatment with rolipram, developed as a potential antidepressant, which was also found to attenuate deficits in MBP in the experimental autoimmune encephalomyelitis (EAE) demyelination animal model^{61,62}. The current findings on MBP suggest that the beneficial effects of LIPUS treatment are, in part, associated with protection of myelin and OLs.

NG2 is an integral membrane proteoglycan found in several progenitor cell populations including OPCs. The loss or lack of OPCs and consequent lack of differentiated OLs is associated with loss of myelination, less support of axons, and subsequent impairment of neurological functions. Expression of NG2 is significantly disturbed in demyelinating animal models including the CPZ exposure⁶³ and EAE models⁶⁴. Previous studies from our group found that this disruption in NG2 expression was attenuated by treatment with quetiapine⁶⁰, fluoxetine (unpublished data) and rTMS (unpublished data). Similarly, the present study found that LIPUS significantly increased the level of NG2 in the brains of mice exposed to CPZ (FIG. 12c). The subsequent IHC staining revealed that NG2-positive cells in mice after LIPUS administration significantly out-numbered those in mice without LIPUS (FIG. 12d). Previous studies have found that NG2-positive cells are altered in depression⁶⁵ and that consecutive ECT treatment dramatically increased the proliferation of NG2-expressing glia cells in animals' hippocampus⁶⁶ and amygdala⁶⁷. The results from the present study support the theory that promotion of OPCs plays a part in the beneficial effects of LIPUS.

This study provided evidence that LIPUS is able to significantly alleviate depressive behaviors in mice after exposure to RRS. In vivo and in vitro results suggest that possible mechanisms for the beneficial effects of LIPUS on depression are promotion of neurogenesis, an increase of BDNF and protection and promotion of myelin and OLs. The findings offer support for LIPUS as a promising therapeutic option for depression.

### EXAMPLES

The following examples are intended to illustrate aspects or features of the claimed invention, but not be limiting of the claimed invention, unless explicitly recited as a limitation.

In one embodiment, the ultrasound transducers are longitudinal wave transducers fabricated by APC International, Ltd, Mackeyville, USA. The incident wave is perpendicular to the surface. Piezoelectric crystal 880 developed by APC has a piezoelectric charge constant d₃₃ of 215 m/V, frequency constants Nₜ of 2110 m/s, and mechanical quality factor of 1000, with 25 mm outside diameters and 12.5 mm length. The larger d₃₃ is, the better the emission performance of the piezoelectric, the stronger the vibration and the ultrasound are [16].

Impedance spectroscopy was carried out using an electrochemical measurement station (SP-200, BioLogic Inc., Seyssinet-Pariset, France). At a fixed frequency of 1.5 MHz, four transducers have almost identical impedance spectroscopy of 97.72 Ohm amplitude and 1.15-degree phase, which is used to design the impedance matching circuit.

Low-intensity pulsed ultrasound device was designed by BINARY lab. The output voltage is an amplitude-adjustable 1.5 MHz square wave signal with 1 kHz repetition rate of 20% duty cycle. Peak-to-peak amplitude vary from 1.25 V to 12.5 V. Incident wave intensity increases with the increase of output signal amplitude.

Polyurethane rubber is cast into a cylindrical shape cap and glued at the front of plastic holder. Grind the rubber to 1.6 mm thickness and fill the gap between transducer and rubber with three different ultrasound coupling agents (purified water, transformer oil and ultrasound gel). The acoustic parameters of three ultrasound agents and polyurethane rubber are presented in Table 1.

TABLE 1
Acoustic•Properties•of•Materials¶

| | Sound• | | Acoustic• | Attenuation• | |
|---|---|---|---|---|---|
| | Velocity• | Density¶ | Impedance¶ | Coefficient¶ | |
| ¤ | mm/s¤ | g/cm^{3¤} | MRayl¤ | dB/cm¤ | [symbol] |
| Water-liquid• | 1.48¤ | 1.00¤ | 1.48¤ | —¤ | [symbol] |
| at•20° C.¤ | | | | | |
| Oil- | 1.39¤ | 0.92¤ | 1.28¤ | —¤ | [symbol] |
| transformer^{a¤} | | | | | |
| Ultrasound• | 1.58¤ | 1.02¤ | 1.61¤ | 0.54• | [symbol] |
| Gel^{b¤} | | | | dB/cm•at•6• | |
| | | | | MHz¤ | |
| Polyurethane• | 2.09¤ | 1.30¤ | 2.36¤ | —¤ | [symbol] |
| Rubber¤ | | | | | |

^{a}Oil-transformer•(100005, •Briggs•&•Stratton•Company, •Milwaukee, •USA)¶
^{b}Ultrasound•Gel•(Aquasonic•100, •Parker•Laboratories, •Inc, •Fairfield, •USA)¶ [symbol]
indicates data missing or illegible when filed

### B. Experiment Methods

As shown in FIG. 4, a LIPUS device is used to generate square waveform of tunable amplitude. The LIPUS device is made in BINARY LAB in Canada and the output amplitude can be controlled by a mobile phone via Bluetooth. Using the above setup, a set of measurements is performed during which the acoustic intensity generated by the ultrasound transducer are read from an ultrasound power meter (Model UPM-DT-1AV from Ohmic Instruments Co., Maryland). These measurements are performed in the vicinity of the nominal resonance frequency of 1.5 MHz. A plastic holder is placed above the ultrasound power meter. By changing the different transducer caps (no cap/sham group, cap filling with water, cap filling with oil and cap filling with gel) on the plastic holder, we obtain the corresponding intensity at the same driving signal. As the amplitude increases, the intensities of different caps increase respectively.

After obtaining the acoustic intensity data, we analysed the attenuation/transmission coefficient (the efficiency of electrical energy into mechanical energy) of each kind of caps using MATLAB. The transmission efficiency was investigated by fitting a linear function between the decayed intensity and the original intensity.

FIG. 5 describes the measured ultrasound intensity and fitting intensity of ultrasound transducers with polyurethane rubber filling with different ultrasound coupling agents comparing to the intensity of a transducer without a cap. Three point-data groups (with error bar) are the intensity measurement data for different ultrasound agents compared to sham group. Each data point represents the average of four times of measurements and standard deviation is calculated to draw the error. Intensity value is obtained by the ultrasound power measured by Ultrasound Power Meter divided by 5 cm\hat{ }2 effective area of the transducers. Due to very low intensity we used in the experiment, ultrasound power can be affected by slight vibration of table or human movement. However, the overall standard deviation for each intensity is very insignificant compared to the intensity value.

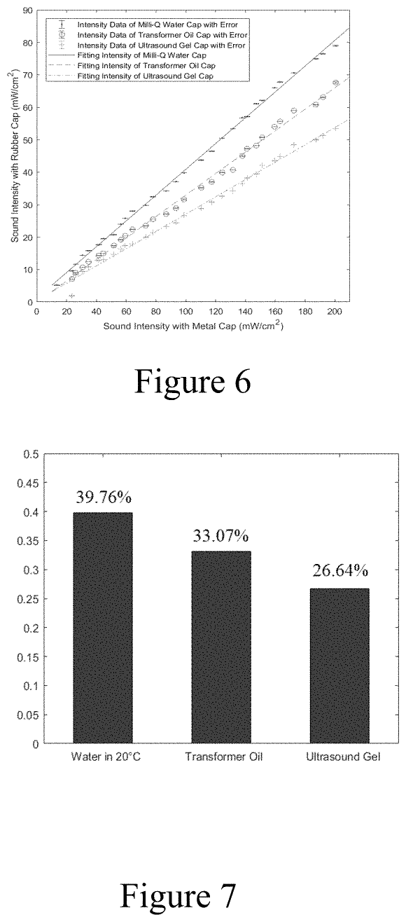

In order to express the intensity attenuation better, ultrasound intensity attenuation linear fitting is then generated by polynomial curve fitting shown in FIG. 6 (solid line, dotted line, and dash-dotted line) for each coupling agent. We choose linear fitting because the attenuation of ultrasound intensity is linear based on the data in FIG. 6. It can easily tell that rubber cap with purified water has a higher slope than other two groups.

Comparisons between three different ultrasound coupling agents (purified water, transformer oil and ultrasound gel) for intensity attenuation have been carried out, as shown in FIG. 6. The results return the coefficients for a polynomial of degree 1 (slope) that is a best fit for various data using MATLAB. Insignificant Bias (polynomial of degree 0) is discarded since all the relationships should start at the coordinate origin.

When the ultrasonic wave is perpendicular to a sufficiently large smooth flat interface, interface transmission wave intensity Iₜ and incident wave intensity I₀ ratio is called transmission coefficient of sound intensity, represented by T [16]:

$$T = \frac{{I}_{t}}{{I}_{i}} = \frac{4 {Z}_{1} {Z}_{2}}{{( {Z}_{2} + {Z}_{1} )}^{2}} , \qquad {( 1 )}^{′}$$

where Z₁ and Z₂ denoted the acoustic impedance of two medium individually. Based on (1), we can see that when acoustic impedance for the incident wave and transmission wave medium (Z₁ and Z₂) is equal or similar, sound intensity of transmission coefficient is close to 1. On the other hand, the greater the acoustic impedance mismatch, the greater the percentage of energy will be reflected at the interface between one medium and another.

Polyurethane rubber cap filling with purified water has a significant higher transmission rate compared to cap filling with oil and ultrasound gel. Theoretically, ultrasound gel has a closest acoustic impedance ρc value in comparison among three mediums, which should lead to a best transmission rate based on (1). Nonetheless, ultrasound gel has a higher attenuation coefficient that cannot be ignored when ultrasound waves transmit through the gel as shown in Table 1. Without restriction to a theory, that might be the main reason why using ultrasound gel as filling medium is turned out to be the worst scenario. The coupling agent which has the second closest acoustic impedance is purified water leading to the best transmission rate of 39.76%.

Experiment Setup

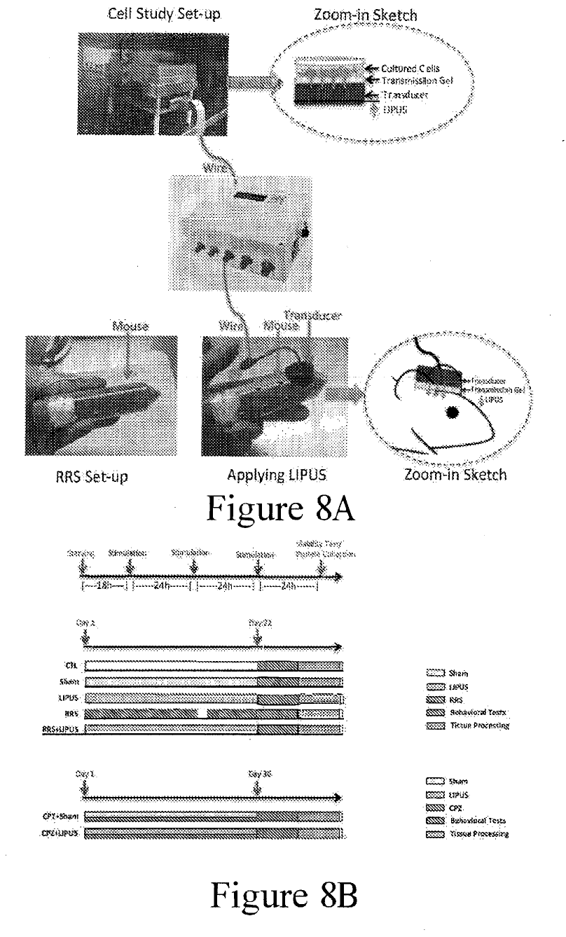

LIPUS was generated using SonaCell™ (IntelligentNano Inc., Edmonton, Canada) (FIG. 8) operating at a frequency of 1.5 MHz and pulse repetition rate of 1.0 kHz. The pulse duty cycle used was 20%. For the cell study, LIPUS was applied by stimulating cells in an enclosed sterile 12-well cell culture plate in an incubator (FIG. 8). Ultrasound was transmitted through the bottom of the wells via transmission gel between the transducer and plate (FIG. 8). Intensity was adjusted to 15 or 30 mW/cm². Treatment was delivered for 5 minutes daily over 3 days.

In the animal study, the intensity was adjusted to 25 mW/cm² and treatment duration was 20 minutes daily. Mice were restrained in plastic tubes with the ultrasound transducer fixed near the end of the tube directly above the mouse's head. Transmission gel was placed between the skin and transducer (FIG. 8). In these studies, whole brain was stimulated.

### Cell Cultures

The SH-SY5Y cell line (Sigma-Aldrich, St. Louis) was cultured on a poly-D-lysine-coated surface at 37° C. with 5% CO₂ and 95% air. Cells were seeded at 1×10⁵ cells/well in 12-well plates with complete fetal bovine serum (FBS)-containing medium and then switched to serum-free DMEM/F-12 medium for 18 hours. Cells were treated with LIPUS at 15 or 30 mW/cm² for 5 minutes daily for 3 sessions. Twenty-four hours after the last treatment, cell viability tests were conducted or cell proteins collected (FIG. 8).

Primary cell cultures were prepared using cerebral cortical glia from 1-day-old rat pups as previously described²¹. Cells were treated with LIPUS at 15 or 30 mW/cm² for 5 minutes daily for 3 sessions. Twenty-four hours after the last stimulation, cell viability tests were conducted or cell proteins collected (FIG. 8).

### Cell Viability Tests

Tests used a cell proliferation reagent colorimetric assay, the Water Soluble Tetrazolium-1 (WST-1) Kit (Roche, Basel), based on manufacturer's instructions. Absorbance was measured at 450 nm.

### Cell Protein Analysis

Samples were collected after LIPUS stimulation using a Tris-ethylenediaminetetraacetic acid (EDTA) lysis buffer (1% Triton X-100, 20 mM Tris, 2 mM EDTA, pH 7.6) with freshly added Protease Inhibitors Cocktail (Sigma-Aldrich). Western blots were conducted as outlined in the following section. Primary antibodies (EMD Millipore Corporation, Darmstadt, Germany) used: rabbit anti-BDNF (dilution 1:500), mouse anti-phosphorylated β-catenin (pβ-catenin) (dilution 1:1000), rabbit anti-β-catenin (dilution 1:500).

### Western Blots

After protein concentration determination with a bicinchoninic acid (BCA) protein assay kit (Thermo Fisher Scientific, Waltham, USA), samples were boiled and loaded (15 μL/well) on SDS-PAGE mini-gels. Proteins were separated by electrophoresis at 80 V for 1.5 hours at room temperature and electrophoretically transferred onto PVDF membranes at 120 mA for 2 hours in ice-cold transfer buffer. Membranes were blocked with 5% (w/v) skim milk in Tris-buffered saline plus Tween-20 (TBST) for 1 hour at room temperature and then incubated with a series of primary antibodies (4° C. overnight). β-Actin was the internal loading control. Membranes were rinsed 3× with TBST and incubated with horseradish peroxidase (HRP)-conjugated secondary antibodies at a dilution of 1:5000 (goat anti-rabbit, Abcam, Cambridge, UK; goat anti-mouse, Cedarlane, Burlington, Canada) for 2 hours at room temperature. Immunoreactive proteins were visualized using an enhanced chemiluminescence (ECL) substrate kit (ThermoFisher Scientific). Quantification of immunoblots was by densitometric analysis of chemiluminescence-exposed films, using the software ImageJ (version 64, NIH, Bethesda), and results expressed as a ratio of target protein to β-actin.

### Animals

Seven-week old female C57BL/6 mice were from Charles River (Montréal). The animal facility was maintained at a 12-hour/12-hour light-dark cycle, at 22±0.5° C., and at 60% humidity. All animal procedures adhered to Canadian Council on Animal Care guidelines and were approved by the University Animal Care and Use Committee.

### Repetitive Restraint Stress (RRS) Administration

After housing-acclimatization for 2 weeks, mice were divided into groups: control (CTL), sham (with the apparatus but transducer not connected to LIPUS generating box), RRS, LIPUS, RRS plus LIPUS (RRS+LIPUS). Mice were fed normal chow. Body weight was measured weekly. RRS was induced by placing the mice in plastic tubes with air holes at the nasal end (FIG. 8) for 3 hours daily for 3 weeks. This procedure is similar to the ultrasound administration in the present study (FIG. 8 for details), providing a suitable experimental set-up to initiate the investigation on the effects of LIPUS with no extra disturbance created by the ultrasound treatment procedure alone. At 24 hours after the last RRS treatment, tests were performed to evaluate depression-like behaviors and brain tissue was then collected (FIG. 8).

### Cuprizone (CPZ) Administration

After acclimatization with normal chow for 2 weeks, mice were divided into CPZ+sham and CPZ+LIPUS groups. CPZ (Sigma-Aldrich) was mixed into milled LabDiet (St. Louis) rodent chow at a concentration of 0.2% (w/w). Mice were fed CPZ food for 5 weeks. Body weight was measured weekly. For the LIPUS group, mice were administered LIPUS for 20 minutes daily. Sham mice were in the LIPUS apparatus for 20 minutes daily, with the cable and generating box not connected. At 24 hours after the last CPZ treatments, tests were performed to evaluate depression-like behaviors, and brain tissue was collected (FIG. 8).

### Behavioral Tests.

The Sucrose Preference Test (SPT), Tail Suspension Test (TST), Forced Swimming Test and Y-Maze Test were conducted as described previously by our group²²⁻²⁴.

### Tissue Processing

Mice were anesthetized with isofluorane and perfused intracardially with 0.01 M PBS, pH 7.4. The brain (excluding cerebellum, pons, and medulla oblongata) was then removed. The right hemisphere was separated into frontal, medial and hind cortex and hippocampus, snap frozen and stored at −80° C. for Western blotting. The left hemisphere was post-fixed in 4% paraformaldehyde (PFA) in PBS for 48 hours, followed by cryoprotection in 30% sucrose (4° C./72 hours). Serial coronal sections (30 μm) were cut by cryostat (Leica Biosystems, Wetzlar, Germany) and collected in 24-well plates containing 0.01 M PBS for IHC staining.

### Animal Protein Analysis

Mice (4-6) were randomly selected from each group, and protein samples from the medial cortex or hippocampus collected using EDTA lysis buffer with added Protease Inhibitors Cocktail. Western blots were done as described in the Western blots section. Primary antibodies used: guinea pig anti-doublecortin (DCX) (EMD Millipore Corporation, Darmstadt, Germany; dilution 1:1000), rabbit anti-BDNF (EMD Millipore Corporation, dilution 1:500), chicken anti-myelin basic protein (MBP) (Ayes Labs, Tigard, USA; dilution 1:2000), rabbit anti-neural/glial antigen 2 (NG2) (EMD Millipore Corporation, dilution 1:1000).

### Immunohistochemistry (IHC) Staining

Free-floating brain sections were washed 3x with PBS and quenched in PBS containing 3% H₂0₂ to block endogenous peroxidase activity. Sections were blocked at room temperature for 60 minutes in 3% normal goat serum, 1% BSA and 0.3% Triton X-100 in PBS and incubated overnight at 4° C. with these primary antibodies: chicken anti-MBP (Ayes Labs, dilution 1:1000), rabbit anti-NG2 (EMD Millipore Corporation, dilution 1:200). Specific secondary biotinylated antibody was incubated at a dilution of 1:400 (goat anti-chicken & goat anti-rabbit, Vector Laboratories, Burlingame, USA) for 1.5 hours at room temperature. After washing 3× with TBST, slides were incubated with avidin-biotin complex (ABC) reagents for 30 minutes. The antigen-antibody complexes were visualized with a diaminobenzidine (DAB) kit (Sigma-Aldrich). Slides were air-dried in the dark, mounted and viewed with a Leica DMI6000B Microscope (Wetzlar) for bright and dark fields and captured with LAS AF computer software. Four or five random fields at 16× (MBP) or 100× (NG2) magnification from each animal of the CPZ+Sham and CPZ+LIPUS groups were examined. Optical densities of the MBP-positive and NG2-positive staining were measured using ImageJ and expressed as relative density.

### Statistical Analysis

The Statistical Package for the Social Sciences (SPSS, version 20, IBM, New York, USA) was used. Results are expressed as means±SEM. Differences across experimental groups were determined by one- or two-way analysis of variance (ANOVA), followed by Newman-Keuls post-hoc tests for multiple comparisons. A two-tailed t-test for independent samples was used for two-group comparisons. A p-value <0.05 was considered statistically significant.

### Results

### Effects of LIPUS on Cell Viability

LIPUS at 15 mW/cm² significantly increased cell viability in SH-SY5Y cells, while 30 mW/cm² did not (FIG. 9a). With primary glia cells, LIPUS at 15 mW/cm² significantly increased cell viability, while 30 mW/cm² stimulation led to a significant decrease in cell viability compared to 15 mW/cm² (FIG. 9b).

### Effects of LIPUS on Wnt Signaling Pathway and BDNF

For neuron-like SH-SY5Y cells, LIPUS at neither 15 mW/cm² or 30 mW/cm² produced no change (showed a non-significant tendency to increase levels of pβ-catenin) (FIG. 9c). For primary glia cells compared to non-stimulated controls, LIPUS at 15 mW/cm² significantly increased the levels of pβ-catenin, while stimulation at 30 mW/cm² did not (FIG. 9d).

With SH-SY5Y cells, LIPUS at both 15 and 30 mW/cm² significantly increased levels of BDNF compared to non-stimulated controls (FIG. 9e). In primary glia cells, LIPUS at 15 mW/cm² significantly increased the level of BDNF while 30 mW/cm² stimulation did not (FIG. 9f).

### Effects of LIPUS on Depression-Like Behaviors in Mice Exposed to RRS

Depression-like behavior was tested with the SPT, TST, FST and YMT. For the SPT, the RRS mice had a significantly lower preference index than the control group and LIPUS treatment significantly increased the preference index in RRS mice (FIG. 10a). There were no significant changes in fluid consumption across groups (FIG. 10b).

In the TST, the RRS+LIPUS mice had a significant decrease of total immobile time compared to the RRS mice (FIG. 10c). In the FST, LIPUS significantly decreased the total time of immobility in the RRS groups (FIG. 10d).

Cognitive deficiency is common in depression²⁵, so spatial working memory was evaluated using the YMT. There were changes in alternation performance, with LIPUS treatment significantly increasing the spontaneous alternations in the RRS mice (FIG. 10e). There were no significant differences in total arm entries across groups (FIG. 10f).

### 3.4 Effects of LIPUS on DCX and BDNF in Mice Exposed to RRS

Analysis of hippocampal proteins indicated that levels of DCX in RRS mouse medial cortex showed a trend toward a significant decrease compared to control mice (p=0.09) and that LIPUS treatment increased the values compared to RRS group (FIG. 11a). LIPUS also significantly increased levels of DCX in control animals (FIG. 11a).

LIPUS significantly increased levels of BDNF in hippocampus of both control mice, and the RRS+LIPUS group had significantly higher BDNF levels than controls and had a non-significant tendency toward higher levels of BDNF than the RRS only group (FIG. 11b).

No Difference in Animal Behaviors Induced by LIPUS after Exposure to CPZ

Mice given CPZ with or without LIPUS were also tested with the TST, FST and YMT. With the TST and FST there was no significant difference in total time of immobility between these two groups. With the YMT, there was no significant difference in spontaneous alterations or in total arm entries between these two groups.

### Effects of LIPUS on the Level of MBP and NG2 in Mice Exposed to CPZ

When comparing cortex of mice from CPZ+Sham and CPZ+LIPUS groups, LIPUS significantly increased levels of MBP in mice exposed to CPZ (FIG. 12a). Subsequent IHC staining showed that LIPUS significantly increased MBP-positive staining in corpus callosum of animals exposed to CPZ (FIG. 12b).

LIPUS significantly increased the level of NG2 in mice exposed to CPZ (FIG. 12c). Subsequent IHC staining showed that LIPUS significantly increased positive staining of NG2 in corpus callosum of animals exposed to CPZ (FIG. 12d).

### Definitions and Interpretation

The description of the present invention has been presented for purposes of illustration and description, but it is not intended to be exhaustive or limited to the invention in the form disclosed. Many modifications and variations will be apparent to those of ordinary skill in the art without departing from the scope and spirit of the invention. Embodiments were chosen and described in order to best explain the principles of the invention and the practical application, and to enable others of ordinary skill in the art to understand the invention for various embodiments with various modifications as are suited to the particular use contemplated.

The corresponding structures, materials, acts, and equivalents of all means or steps plus function elements in the claims appended to this specification are intended to include any structure, material, or act for performing the function in combination with other claimed elements as specifically claimed.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such aspect, feature, structure, or characteristic with other embodiments, whether or not explicitly described. In other words, any element or feature may be combined with any other element or feature in different embodiments, unless there is an obvious or inherent incompatibility between the two, or it is specifically excluded.

It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition or step being referred to is an optional (not required) feature of the invention.

The singular forms “a,” “an,” and “the” include the plural reference unless the context clearly dictates otherwise. The term “and/or” means any one of the items, any combination of the items, or all of the items with which this term is associated.

As will be understood by the skilled artisan, all numbers, including those expressing quantities of reagents or ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

### REFERENCES

The following references are provided as indicative of the level of skill in the art, and are incorporated herein by reference in their entirety, for all purposes, except where prohibited.

Ensminger, D. and L. J. Bond, Ultrasonics: fundamentals, technologies, and applications. 2011: CRC press.

Khanna, A., et al., The effects of LIPUS on soft-tissue healing: a review of literature. British Medical Bulletin, 2009. 89(1): p. 169-182.

Malizos, K. N., et al., Low-intensity pulsed ultrasound for bone healing: An overview. Injury, 2006. 37(1): p. S56-S62.

El-Bialy, T., I. El-Shamy, and T. M. Graber, Repair of orthodontically induced root resorption by ultrasound in humans. American Journal of Orthodontics and Dentofacial Orthopedics, 2004. 126(2): p. 186-193.

Al-Daghreer, S., et al., Long term effect of Low Intensity Pulsed Ultrasound on a human tooth slice organ culture. Archives of Oral Biology, 2012. 57(6): p. 760-768.

Xin, Z., et al., Clinical applications of low-intensity pulsed ultrasound and its potential role in urology. Translational Andrology and Urology, 2016. 5(2): p. 255-266.

Huang, S.-L., et al., Protective Effect of Low-Intensity Pulsed Ultrasound on Memory Impairment and Brain Damage in a Rat Model of Vascular Dementia. Radiology, 2017. 282(1): p. 113-122.

Werner-Seidler, A., et al., School-based depression and anxiety prevention programs for young people: A systematic review and meta-analysis. Clinical Psychology Review, 2017. 51: p. 30-47.

Marcus, Sheila M.; Flynn, Heather A.; Blow, Frederic C.; Barry, Kristen L. (2003). “Depressive Symptoms among Pregnant Women Screened in Obstetrics Settings.” Journal of Women's Health 12(4): 373-380

Tyler, W. J., et al., Remote Excitation of Neuronal Circuits Using Low-Intensity, Low-Frequency Ultrasound. PLoS ONE, 2008. 3(10): p. e3511.

Tufail, Y., et al., Transcranial Pulsed Ultrasound Stimulates Intact Brain Circuits. Neuron, 2010. 66(5): p. 681-694.

Eriksson, T. J. R., S. M. Dixon, and S. N. Ramadas. Flexural mode metal cap transducer design for specific frequency air coupled ultrasound generation. in 2013 IEEE International Ultrasonics Symposium (IUS). 2013.

Ketterling, J. A., Acoustic field of a wedge-shaped section of a spherical cap transducer. The Journal of the Acoustical Society of America, 2003. 114(6): p. 3065-3075.

Garu, P. K. and T. K. Chaki, Acoustic and mechanical properties of neoprene rubber for encapsulation of underwater transducers. Intl. J. of Scientific Engineering and Technology, 2012. 1(5): p. 231-237.

Mott, P. H., C. M. Roland, and R. D. Corsaro, Acoustic and dynamic mechanical properties of a polyurethane rubber. The Journal of the Acoustical Society of America, 2002. 111(4): p. 1782-1790.

Blitz, J., Fundamentals of Ultrasonics. 1963.
1. Rush, A J, Trivedi M H, Wisniewski S R, Stewart J W, Nirenberg A A, Thase M E et al. Bupropion-SR, sertraline, or venlafaxine-XR after failure of SSRIs for depression. N Engl J Med 2006; 354(12): 1231-1242.
2. Shankar H, Pagel P S. Potential adverse ultrasound-related biological effects: a critical review. Anesthesiology 2011; 115(5): 1109-1124.
3. Tyler W J, Tufail Y, Finsterwald M, Tauchmann M L, Olson E J, Majestic C. Remote excitation of neuronal circuits using low-intensity, low-frequency ultrasound. PLoS One 2008; 3(10): e3511.
4. Tufail Y, Matyushov A, Baldwin N, Tauchmann M L, Georges J, Yoshihiro et al. Transcranial pulsed ultrasound stimulates intact brain circuits. Neuron 2010; 66(5): 681-694.
5. Scarcelli T, Jordao J F, O'Reilly M A, Ellens N, Hynynen K, Aubert I. Stimulation of hippocampal neurogenesis by transcranial focused ultrasound and microbubbles in adult mice. Brain Stimul 2014; 7(2): 304-307.
6. McKinnon M C, Yucel K, Nazarov A, MacQueen G M. A meta-analysis examining clinical predictors of hippocampal volume in patients with major depressive disorder. J Psychiatry Neurosci 2009: 34(1): 41-54.
7. Otsuki K, Uchida S, Watanuki T, Wakabayashi Y, Fujimoto M, Matsubara T et al. Altered expression of neurotrophic factors in patients with major depression. J Psychiatr Res 2008; 42(14): 1145-1153.
8. Hayley S, Poulter M O, Merali Z, Ainisman H. The pathogenesis of clinical depression: stressor- and cytokine-induced alterations of neuroplasticity. Neuroscience 2005; 135(3): 659-678.
9. Hameroff S, Trakas M, Duffield C, Annabi E, Gerace M B, Boyle P et al. Transcranial ultrasound (TUS) effects on mental states: a pilot study. Brain Stimul 2013; 6(3): 409-415.
10. Lie D C, Colamarino S A, Song H J, Desire L, Mira H, Consiglio A et al. Wnt signalling regulates adult hippocampal neurogenesis. Nature 2005; 437(7063): 1370-1375.
11. De Ferrari G V, Moon R T. The ups and downs of Wnt signaling in prevalent neurological disorders. Oncogene 2006. 25(57): 7545-7553.
12. Inkster B, Nichols T E, Saemann P G, Auer D P, Holsboer F, Muglia P et al. Pathway-based approaches to imaging genetics association studies: Wnt signaling, GSK3beta substrates and major depression. Neuroimage 2010; 53(3): 908-917.
13. Magavi S S, Leavitt B R, Macklis J D. Induction of neurogenesis in the neocortex of adult mice. Nature 2000; 405(6789): 951-955.
14. Matrisciano F, Bonaccorso S, Ricciardi A, Scaccianoce S, Panaccione I, Wang L et al. Changes in BDNF serum levels in patients with major depression disorder (MDD) after 6 months treatment with sertraline, escitalopram, or venlafaxine. J Psychiatr Res 2009; 43(3): 247-254.
15. Molteni R, Calabrese F, Cattaneo A, Mancini M, Gennarelli M, Racagni G et al. Acute stress responsiveness of the neurotrophin BDNF in the rat hippocampus is modulated by chronic treatment with the antidepressant duloxetine. Neuropsychopharmacology 2009; 34(6): 1523-1532.

016. Doron R, Lotan D, Versano Z, Benatav L, Franko M, Armoza S et al. Escitalopram or novel herbal mixture treatments during or following exposure to stress reduce anxiety-like behavior through corticosterone and BDNF modifications. PLoS One 2014; 9(4): e91455.
17. Engel D, Zomkowski A D, Lieberknecht V, Rodrigues A L, Gabilan N H. Chronic administration of duloxetine and mirtazapine downregulates proapoptotic proteins and upregulates neurotrophin gene expression in the hippocampus and cerebral cortex of mice. J Psychiatr Res 2013; 47(6): 802-808.
18. Freire T F, Fleck M P, da Rocha N S. Remission of depression following electroconvulsive therapy (ECT) is associated with higher levels of brain-derived neurotrophic factor (BDNF). Brain Res Bull 2016; 121: 263-269.
19. Gedge L, Beaudoin A, Lazowski L, du Toit R, Jokic R, Milev R. Effects of electroconvulsive therapy and repetitive transcranial magnetic stimulation on serum brain-derived neurotrophic factor levels in patients with depression. Front Psychiatry 2012; 3: 12.
20. Wang H, Xu H, Niu J, Mei F, Li X, Kong J et al., Haloperidol activates quiescent oligodendroglia precursor cells in the adult mouse brain. Schizophr Res 2010: 119(1-3): 164-174.
21. Zhu S, Shi R, Wang J, Wang J F, Li X M. Unpredictable chronic mild stress not chronic restraint stress induces depressive behaviours in mice. Neuroreport 2014; 25(14): 1151-1155.
22. Yan B, He J, Xu H, Zhang Y, Bi X, Thakur S et al. Quetiapine attenuates the depressive and anxiolytic-like behavioural changes induced by global cerebral ischemia in mice. Behav Brain Res 2007; 182(1): 36-41.
23. Wang J, Qiao J, Zhang Y, Wang H, Zhu S, Zhang H et al., Desvenlafaxine prevents white matter injury and improves the decreased phosphorylation of the rate-limiting enzyme of cholesterol synthesis in a chronic mouse model of depression. J Neurochem 2014; 131(2): 229-238.
24. Zhang H, Zhang Y, Xu H, Wang L, Zhao J, Wang J et al. Locomotor activity and anxiety status, but not spatial working memory, are affected in mice after brief exposure to cuprizone. Neurosci Bull 2013; 29(5): 633-641.
25. Papakostas G I. Cognitive symptoms in patients with major depressive disorder and their implications for clinical practice. J Clin Psychiatry 2014; 75(1): 8-14.
26. Bachtold M R, Rinaldi P C, Jones J P, Reines F, Price L R. Focused ultrasound modifications of neural circuit activity in a mammalian brain. Ultrasound Med Biol 1998; 24(4): 557-565.
27. King R L, Brown J R, Newsome W T, Pauly K B. Effective parameters for ultrasound-induced in vivo neurostimulation. Ultrasound Med Biol 2013; 39(2): 312-331.
28. Legon W, Sato T F, Opitz A, Mueller J, Barbour A, Williams A et al. Transcranial focused ultrasound modulates the activity of primary somatosensory cortex in humans. Nat Neurosci 2014; 17(2): 322-329.
29. Xu P, Gul-Uludag H, Ang W T, Yang X, Huang M, Marquez-Curtis Let al. Low-intensity pulsed ultrasound-mediated stimulation of hematopoietic stem/progenitor cell viability, proliferation and differentiation. Biotechnol Lett 2012; 34(10): 1965-1973.
30. Zhao Y, Xing J, Xing J Z, Ang W T, Chen J. Applications of low-intensity pulsed ultrasound to increase monoclonal antibody production in CHO cells using shake flasks or wavebags. Ultrasonics 2014; 54(6): 1439-1447.
31. Leung K S, Lee W S, Tsui H F, Liu P P, Cheung W H. Complex tibial fracture outcomes following treatment with low-intensity pulsed ultrasound. Ultrasound Med Biol 2004; 30(3): 389-395.
32. Kuroda Y, McEwen B S. Effect of chronic restraint stress and tianeptine on growth factors, growth-associated protein-43 and microtubule-associated protein 2 mRNA expression in the rat hippocampus. Brain Res Mol Brain Res 1998; 59(1): 35-39.
33. Yamaura K, Bi Y, Ishiwatari M, Oishi N, Fukata H, Ueno K. Sex differences in stress reactivity of hippocampal BDNF in mice are associated with the female preponderance of decreased locomotor activity in response to restraint stress. Zoolog Sci 2013; 30(12): 1019-1024.
34. Maghsoudi N, Ghasemi R, Ghaempanah Z, Ardekani M, Nooshinfar E, Tahzibi A. Effect of chronic restraint stress on HPA axis activity and expression of BDNF and Trkb in the hippocampus of pregnant rats: Possible contribution in depression during pregnancy and postpartum period. Basic Clin Neurosci 2014; 5(2): 131-137.
35. Willner P. Chronic mild stress (CMS) revisited: consistency and behavioural-neurobiological concordance in the effects of CMS. Neuropsychobiology 2005; 52(2): 90-110.
36. Mateus-Pinheiro A, Patricio P, Alves N D, Machado-Santos A R, Morais M, Bessa J M et al. The Sweet Drive Test: refining phenotypic characterization of anhedonic behavior in rodents. Front Behav Neurosci 2014; 8: p. 74.
37. Hageman I, Nielsen M, Wortwein G, Diemer N H, Jorgensen M B. Electroconvulsive stimulations normalizes stress-induced changes in the glucocorticoid receptor and behaviour. Behav Brain Res 2009; 196(1): 71-77.
38. Ampuero E, Luarte A, Santibanez M, Varas-Godoy M, Toledo J, Diaz-Veliz Get al. Two chronic stress models based on movement restriction in rats respond selectively to antidepressant drugs: Aldolase C as a potential biomarker. Int J Neuropsychopharmacol 2015;

18(10): pyv038.
39. Castagne V, Moser P, Roux S, Porsolt R D. Rodent models of depression: forced swim and tail suspension behavioral despair tests in rats and mice. Curr Protoc Neurosci 2011; Chapter 8: Unit 8 10A.
40. Griebel G, Cohen C, Perrault G, Sanger D J. Behavioral effects of acute and chronic fluoxetine in Wistar-Kyoto rats. Physiol Behav 1999; 67(3): 315-320.
41. Berrocoso E, Mico J A. Role of serotonin 5-HT1A receptors in the antidepressant-like effect and the antinociceptive effect of venlafaxine in mice. Int J Neuropsychopharmacol 2009; 12(1): 61-71.
42. Bourin M, Chenu F, Ripoli N, David D J. A proposal of decision tree to screen putative antidepressants using forced swim and tail suspension tests. Behav Brain Res 2005; 164(2): 266-269.
43. Kotagale N R, Mendhi S M, Aglawe M M, Umekar M J, Taksande B G. Evidences for the involvement of sigma receptors in antidepressant like effect of quetiapine in mice. Eur J Pharmacol 2013; 702(1-3): 180-186.
44. Teste J F, Martin I, Rinjard P. Electrotherapy in mice: dopaminergic and noradrenergic effects in the Tail Suspension Test. Fundam Clin Pharmacol 1990; 4(1): 39-47.
45. Gur E, Lerer B, van de Kar L D, Newman M E. Chronic rTMS induces subsensitivity of post-synaptic 5-HT1A receptors in rat hypothalamus. Int J Neuropsychopharmacol 2004; 7(3): 335-340.
46. Hamani C, Diwan M, Isabella S, Lozano A M, Nobrega J N. Effects of different stimulation parameters on the antidepressant-like response of medial prefrontal cortex deep brain stimulation in rats. J Psychiatr Res 2010; 44(11): 683-687.
47. Czeh B, Michaelis T, Watanabe T, Frahm J, de Birrun G, van Kampen M et al. Stress-induced changes in cerebral metabolites, hippocampal volume, and cell proliferation are prevented by antidepressant treatment with tianeptine. Proc Natl Acad Sci USA 2001; 98(22): 12796-12801.
48. Petrik D, Lagace D C, Eisch A J, The neurogenesis hypothesis of affective and anxiety disorders: are we mistaking the scaffolding for the building? Neuropharmacology 2012; 62(1): 21-34.
49. Peng Q, Masuda N, Jiang M, Li Q, Zhao M, Ross C A et al. The antidepressant sertraline improves the phenotype, promotes neurogenesis and increases BDNF levels in the R6/2 Huntington's disease mouse model. Exp Neurol 2008; 210(1): 154-163.
50. Madsen T M, Treschow A, Bengzon J, Bolwig T G, Lindvall O, Tingstrom A. Increased neurogenesis in a model of electroconvulsive therapy. Biol Psychiatry 2000; 47(12): 1043-1049.
51. Ueyama E, Ukai S, Ogawa A, Yamamoto M, Kawaguchi S, Ishii R et al. Chronic repetitive transcranial magnetic stimulation increases hippocampal neurogenesis in rats. Psychiatry Clin Neurosci 2011; 65(1): 77-81.
52. Ohira K, Takeuchi R, Shoji H, Miyakawa T. Fluoxetine-induced cortical adult neurogenesis. Neuropsychopharmacology 2013; 38: 909-920.
53. Jaako-Movits K, Zharkovsky T, Pedersen M, Zharkovsky A. Decreased hippocampal neurogenesis following olfactory bulbectomy is reversed by repeated citalopram administration. Cell Mol Neurobiol 2006; 26(7/8): 1559-1570.
54. Edgar N, Sibille E. A putative functional role for oligodendrocytes in mood regulation. Transl Psychiatry 2012; 2: e109.
55. Hemanth Kumar B S, Mishra S K, Trivedi R, Singh S, Rana P, Khushu S. Demyelinating evidences in CMS rat model of depression: A DTI study at 7T. Neuroscience 2014; 275: 12-21.
56. Murphy R, O'Donoghue S, Counihan T, McDonald C, Calabresi P A, Ahmed M A S. Neuropsychiatric syndromes of multiple sclerosis. J Neurol Neurosurg Psychiatry, 2017; (in press).
57. Hibbits, N, Pannu R, Wu T J, Armstrong, R C., Cuprizone demyelination of the corpus callosum in mice correlates with altered social interaction and impaired bilateral sensorimotor coordination. ASN Neuro, 2009; 1(3): 153-164.
58. Xiao L, Xu H, Zhang Y, Wei Z, He J, Jian W et al. Quetiapine facilitates oligodendrocyte development and prevents mice from myelin breakdown and behavioral changes. Mol Psychiatry 2008; 13(7): 697-708.
59. Zhang Y, Xu H, Jiang W, Xiao L, Yan B, He J et al. Quetiapine alleviates the cuprizone-induced white matter pathology in the brain of C57BL/6 mouse. Schizophr Res 2008; 106(2-3): 182-191.
60. Zhang Y, Zhang H, Wang L, Jiang W, Xu H, Xiao L et al. Quetiapine enhances oligodendrocyte regeneration and myelin repair after cuprizone-induced demyelination. Schizophr Res 2012. 138(1): 8-17.
61. Jung S, Zielasek J, Kollner G, Donhauser T, Toyka K, Hartung H P. Preventive but not therapeutic application of Rolipram ameliorates experimental autoimmune encephalomyelitis in Lewis rats. J Neuroimmunol 1996; 68(1-2): 1-11.
62. Gold R., Hartung H P, Toyka K V Animal models for autoimmune demyelinating disorders of the nervous system. Mol Med Today 2000. 6(2): 88-91.
63. Tsiperson S, Huang Y, Bagayogo I, Song Y, VonDran M W, DiCicco-Bloom E, Dreyfus C F. Brain-derived neurotrophic factor deficiency restricts proliferation of oligodendrocyte progenitors following cuprizone-induced demyelination. ASN Neuro 2015; 7(1): 1-11.
64. Tripathi R B, Rivers L E, Young K M, Jamen F, Richardson W D. NG2 glia generate new oligodendrocytes but few astrocytes in a murine experimental autoimmune encephalomyelitis model of demyelinating diseases. J Neurosci 2010; 30(48): 16383-16390.
65. Elsayed M, Banasr M, Duric V, Fournier N M, Licznerski P, Duman R S. Antidepressant effects of fibroblast growth factor-2 in behavioral and cellular models of depression. Biol Psychiatry 2012. 72(4): 258-265.
66. Wennstrom M, Hellsten J, Ekdahl C T, Tingstrom A. Electroconvulsive seizures induce proliferation of NG2-expressing glial cells in adult rat hippocampus. Biol Psychiatry 2003; 54(10): 1015-1024.
67. Wennstrom M, Hellsten J, Tingstrom A. Electroconvulsive seizures induce proliferation of NG2-expressing glial cells in adult rat amygdala. Biol Psychiatry 2004. 55(5): 464-471.

1. A portable LIPUS therapy device for treating mental health disorders, syndromes or diseases, comprising:
(a) a wearable headband comprising at least one transducer element, comprising a piezoelectric crystal, a liquid coupling medium, and an elastomeric cap;
(b) a controller for causing the transducer to produce pulsed ultrasound having an intensity of between about 20 mW/cm2 to about 150 mW/cm2, at a frequency between about 1.0 MHz to about 2.0 MHz, with a pulse repetition rate of between about 0.5 kHz to about 2.0 kHz, and duty cycle between about 10% and 50%.

2. The device of claim 1 wherein the liquid coupling medium comprises purified water.

3. The device of claim 1 wherein the elastomeric cap comprise polyurethane rubber.

4. The device of claim 1, comprising two transducer elements, positioned on the headband to contact the left and right temples of a user wearing the headband.

5. A method of treating a mental health disorder, syndrome or disease in a mammal, comprising the step of applying LIPUS therapy device to the mammals cranium, with a wearable headband comprising at least one transducer element, comprising a piezoelectric crystal, a liquid coupling medium, and an elastomeric cap.

6. The method of claim 5 wherein the transducer element is controlled by a controller configured to cause the transducer to produce pulsed ultrasound having an intensity of between about 20 mW/cm2 to about 150 mW/cm2, at a frequency between about 1.0 MHz to about 2.0 MHz, with a pulse repetition rate of between about 0.5 kHz to about 2.0 kHz, and duty cycle between about 10% and 50%.

7. The method of claim 5 wherein the treatment intensity is about 25 mW/cm² and treatment duration is about 20 minutes daily.

8. The method of claim 5 wherein the mammal is a human and the mental health disorder, syndrome or disease is depression.

9. The method of claim 5 comprising the step of enhancing hippocampal neurogenesis.
